(19) United States
(12) Patent Application Publication
(10) Pub. No.: US 2025/0292911 A1
(43) Pub. Date: Sep. 18, 2025

(54) MULTI-MODAL BRAIN NETWORK COMPUTATION METHOD ASSOCIATED WITH STRUCTURAL FUNCTION APPARATUS, DEVICE, AND MEDIUM

(71) Applicant: SHENZHEN INSTITUTES OF ADVANCED TECHNOLOGY, SHENZHEN (CN)

(72) Inventors: SHUQIANG WANG, SHENZHEN (CN); JUNREN PAN, SHENZHEN (CN)

(21) Appl. No.: 19/220,112

(22) Filed: May 28, 2025

Related U.S. Application Data
(63) Continuation of application No. PCT/CN2022/134902, filed on Nov. 29, 2022.

(51) Int. Cl. G16H 50/30; A61B 5/00; A61B 5/055; G06N 3/094; G06T 7/00

(52) U.S. Cl. CPC G16H 50/30; A61B 5/0042; A61B 5/055; A61B 5/4842; G06N 3/094; G06T 7/0012; G06T 2207/10088; G06T 2207/20081; G06T 2207/30016

(57) ABSTRACT

The present disclosure relates to a multi-modal brain network computation method associated with structural function, apparatus, device, and medium. The method is applied to train a brain disease prediction model, and the brain disease prediction model includes an association perception dual-channel generation module, a disease feature regression module, a topological structure discriminator, and a time-space joint discriminator. In a model training process, by performing a multi-level interactive fusion learning on a high-order topological feature of brain functional magnetic resonance data and magnetic resonance diffusion tensor imaging data, a multi-modal time series activity signal of each brain region is obtained.

### CROSS-REFERENCE TO RELATED APPLICATIONS

This application is a continuation of co-pending International Patent Application Number PCT/CN2022/134902, filed on Nov. 29, 2022, the disclosure of which is incorporated herein by reference in its entirety.

### TECHNICAL FIELD

The present disclosure relates to machine learning technology field, and particularly to a multi-modal brain network computation method associated with structural function, apparatus, device, and medium.

### BACKGROUND

At present, brain diseases have become a common health problem in the world. It seriously endangers life safety of patients. Therefore, more and more attention has been paid to detection and diagnosis of the brain disease. Research direction of brain connection is one of these aspects. By analyzing the brain connection, diagnosis and pathologic traceability of neurodegenerative diseases are facilitated. Taking Alzheimer's disease (AD) as an example, the patients with AD will have brain connection changes during the development of the disease. These changing features may be obtained by brain images such as functional magnetic resonance imaging (FMRI) and diffusion tensor imaging (DTI). In a conventional method, a professional physician sets specific parameters, manually registers, and image corrects to obtain an effective connectivity by a software template. This traditional pathologic feature analysis method highly depends on an experience of the professional physician, which has high time cost and labor cost, and an output effect is greatly affected by parameters setting of the software template, and is not conducive to personalized accurate diagnosis and treatment.

With the development of artificial intelligence technologies, there have a large number of brain connection intelligent computing systems that do not depend on the professional physician. The effectively connected intelligent computing system can be divided into two categories: 1) effective connectivity intelligent computation based on single-model and 2) effective connectivity intelligent computation based on multi-modal. However, the above-mentioned single-mode signal mainly reflects activity features of the brain region, and a main defect is that neural fiber structure features between the brain regions are missing, which leads to the inability to use overall brain topology information to guide a directional causal relationship between brain regions. Thus, a learning capability and precision of the model are limited. On the other hand, an existing intelligent commutating system based on the multi-modal brain neural image data only fuses the multi-modal brain data in an affine splicing or weighted summation manner. A problem of these methods is that heterogeneity-heterogeneity of different modal data is ignored. Therefore, complementary information between different modes is difficult to be deeply excavated, which limits a performance of the model, and resulting in poor practicability and low accuracy of the final model.

### SUMMARY

The present disclosure provides a multi-modal brain network computation method associated with structural function, apparatus, device, and medium, to resolve a problem of a poor practicality and a low precision of a conventional brain disease prediction model.

To resolve the foregoing problem, a technical solution used in this disclosure is that: providing a multi-modal brain network computation method associated with structural function, wherein the method is applied to train a brain disease prediction model, and the brain disease prediction model comprises an association perception dual-channel generation module, a disease feature regression module, a topological structure discriminator, and a time-space joint discriminator; the method comprising: acquiring brain functional magnetic resonance data and magnetic resonance diffusion tensor imaging data; inputting the brain functional magnetic resonance data and the magnetic resonance diffusion tensor imaging data into the association perception dual-channel generation module to perform an interactive association perception fusion and obtain a multi-modal brain activity signal feature, a multi-modal effective connectivity matrix, and a reconstructed structural connectivity matrix; inputting the multi-modal effective connectivity matrix into the disease feature regression module for prediction, inputting the reconstructed structural connectivity matrix into the topological structure discriminator for prediction, and inputting the multi-modal brain region activity signal feature into the time-space joint discriminator for prediction; and according to a predicted result and a pre-constructed loss function, reversely updating the association perception dual-channel generation module, the disease feature regression module, the topological structure discriminator, and the time-space joint discriminator.

As a further improvement of this disclosure, the association perception dual-channel generation module comprises a brain region feature extraction module, a structure-to-function conversion module, a function-to-structure conversion module, a directional global causal inference module, and a structure decoding module.

As a further improvement of this disclosure, inputting the brain functional magnetic resonance data and the magnetic resonance diffusion tensor imaging data into the association perception dual-channel generation module to perform an interactive association perception fusion and obtain a multi-modal brain activity signal feature, a multi-modal effective connectivity matrix, and a reconstructed structural connectivity matrix, comprises: separately extracting a first initial feature and a second initial feature from the brain functional magnetic resonance data and the magnetic resonance diffusion tensor imaging data by the brain region feature extraction module; and inputting the first initial feature and the second initial feature into the structure-to-function conversion module, performing a weighted fusion on a feature output from the structure-to-function conversion module and the first initial feature, to obtain a new first initial feature, and repeat this step to finally obtain the multi-modal brain region activity signal feature; and inputting the first initial feature and the second initial feature into the function-to-structure conversion module, performing a weighted fusion on a feature output from the function-to-structure conversion module and the second initial feature to obtain a new second initial feature, and repeat this step to finally obtain the multi-modal structure feature; and inputting the multi-modal brain region activity signal feature into the directional global causal inference module to obtain the multi-modal brain effective connectivity matrix, and inputting the multi-modal brain structure feature to the structure decoding module to obtain the reconstructed structural connectivity matrix.

As a further improvement of this disclosure, inputting the brain functional magnetic resonance data and the magnetic resonance diffusion tensor imaging data into the association perception dual-channel generation module to perform an interactive association perception fusion and obtain a multi-modal brain activity signal feature, a multi-modal effective connectivity matrix, and a reconstructed structural connectivity matrix, comprises: separately extracting a first initial feature and a second initial feature from the brain functional magnetic resonance data and the magnetic resonance diffusion tensor imaging data by the brain region feature extraction module; inputting the first initial feature and the second initial feature into the structure-to-function conversion module, performing a weighted fusion on a feature output from the structure-to-function conversion module and the first initial feature, to obtain a new first initial feature, and repeat this step to finally obtain the multi-modal brain region activity signal feature; inputting the first initial feature and the second initial feature into the function-to-structure conversion module, performing a weighted fusion on a feature output from the function-to-structure conversion module and the second initial feature to obtain a new second initial feature, and repeat this step to finally obtain the multi-modal structure feature; and inputting the multi-modal brain region activity signal feature into the directional global causal inference module to obtain the multi-modal brain effective connectivity matrix, and inputting the multi-modal brain structure feature to the structure decoding module to obtain the reconstructed structural connectivity matrix. the inputting the multi-modal brain effective connectivity matrix into the disease feature regression module for prediction, inputting the reconstructed structural connectivity matrix into the topological structure discriminator for prediction, and inputting the multi-modal brain region activity signal feature into the time-space joint discriminator for prediction, comprises: inputting the multi-modal brain effective connectivity matrix into the disease feature regression module for prediction, to obtain a disease state prediction probability; inputting the reconstructed structural connectivity matrix and an empirical structural connectivity matrix output by a pre-processing software template into the topological structure discriminator for prediction, to obtain a probability that the reconstructed structural connectivity matrix is output by the association perception dual-channel generation module or output by the pre-processing software template; and inputting the multi-modal brain activity signal feature and an empirical blood oxygen signal output by the pre-processing software template into the time-space joint discriminator for prediction, to obtain a probability that the multi-modal brain activity signal feature is output by the association perception dual-channel generation module or output by the pre-processing software template.

As a further improvement of this disclosure, the time-space joint discriminator comprises a time difference discriminating module and a spatial phase discriminating module, the time difference discriminating module is configured to constrain the association perception dual-channel generation module from a time continuity feature of a brain region activity time series signal, and the spatial phase discriminating module constrains the association perception dual-channel generation module from a spatial field distribution of the brain region activity signal.

As a further improvement of this disclosure, the loss function comprises a disease feature regression loss, a topological adversarial loss, a topological perception loss, a time-space joint adversarial loss, and an attribution measure constraint loss;

The disease feature regression loss is configured to guide the disease feature regression module and the association perception dual-channel generation module to update parameters, and the disease feature regression loss is represented as:

$${ℒ}_{cls} = {𝔼}_{y} [ - log {p}_{c} ( y \vert A ) ] ;$$

Wherein, [symbol] _{cls} represents the disease feature regression loss, A represents the multi-modal effective connectivity matrix, y represents a disease state, p_{c}(y|A) represents the disease state prediction probability, [symbol] _{y} represents an expectation of the disease state probability predicted by the model in a real label distribution, and is used as a loss function for guiding the model to learn.

The topological adversarial loss is configured to guide the topological structure discriminator and the association perception dual-channel generation module to update parameters, and the topological adversarial loss is represented as:

$${ℒ}_{top}^{D} = 𝔼 [ log {D}_{top} ( {S}^{′} ) ] + 𝔼 [ log ( 1 - {D}_{top} ( S ) ) ] ;$$

$${ℒ}_{top}^{G} = 𝔼 [ log {D}_{top} ( S ) ] ;$$

Wherein, [symbol] ₜₒₚ^{D} represents a loss function that guides the topological structure discriminator to learn, [symbol] ₜₒₚ^{G} represents a loss function that guides the generator to learn by the topological structure discriminator, S represents the reconstructed structural connectivity matrix, S′ represents the empirical structural connectivity matrix that is output by the pre-processing software template, and Dₜₒₚ represents the topological structure discriminator;

The topological perception loss is configured to guide the association perception dual-channel generation module to update parameters, and the topological perception loss is represented as:

$${ℒ}_{a w a} = {‖ S - {S}^{′} ‖}_{2} + λ {‖ {SS}^{T} - ( {S}^{′} ) {( {S}^{′} )}^{T} ‖}_{2} ;$$

Wherein, [symbol] _{awa} represents the topological perception loss, ∥·∥₂ represents a Frobenius norm of a matrix, and λ represents a preset super parameter;

The time-space joint adversarial loss is configured to guide the time difference discriminating module, the space phase discriminating module, and the association perception dual-channel generation module to update parameters, and the time-space joint adversarial loss is represented as:

$${ℒ}_{uni}^{D} = 𝔼 [ log ( \frac{1}{2} {D}_{tmp} ( {S}^{′} ) + \frac{1}{2} {D}_{spa} ( {S}^{′} ) ) ] + 𝔼 [ log ( 1 - \frac{1}{2} {D}_{tmp} ( S ) - \frac{1}{2} {D}_{spa} ( S ) ] ;$$

$${ℒ}_{uni}^{G} = 𝔼 [ log ( \frac{1}{2} {D}_{tmp} ( S ) + \frac{1}{2} {D}_{spa} ( S ) ) ] ;$$

Wherein, [symbol] ᵤₙᵢ^{D} represents a loss function for guiding the time-space joint discriminator to learn, [symbol] ᵤₙᵢ^{G} represents a loss function for guiding the generator to learn by the time-space joint discriminator, Dₜₘₚ represents the time difference discriminating module, and Dₛₚₐ represents the space phase discriminating module;

The attributive metric constraint loss is configured to guide the association perception two-channel generation module to update parameter, and the attributive metric constraint loss is represented as:

$$ℒ ( A , B ) = {‖ A \cdot AB - 2 AB + B ‖}_{2} ;$$

Wherein, [symbol] (A,B) represents the attributive metric constraint loss, B represents the [symbol] multi-modal brain region activity signal feature.

As a further improvement of this disclosure, the topological structure discriminator comprises a multi-layer nonlinear topological perception network and a fully connection layer, and an updating formula of the multi-layer nonlinear topological perception network is represented as:

$${F}^{( l + 1 )} = σ ( {D}^{- \frac{1}{2}} \cdot S \cdot {D}^{- \frac{1}{2}} {F}^{( l )} {W}^{( l )} + {b}^{( l )} ) ;$$

Wherein, S represents the reconstructed structural connectivity matrix, D represents a weighted dispersion matrix corresponding to the reconstructed structural connectivity matrix, F^{(l)} represents a topological feature of the lth layer, F^{(l+1)} represents a topological feature of the (l+1)th layer, W^{(l)} is a learnable weight matrix of the lth layer, b^{(l)} is a learnable nonlinear deviation of the lth layer, and σ represents a sigmoid activation function.

To resolve the foregoing technical problem, another technical solution used in this disclosure is that: providing a multi-modal brain network computation device associated with structural function, comprising: an acquiring module, configured to acquire brain functional magnetic resonance data and magnetic resonance diffusion tensor imaging data; a fusion module, configured to input the brain functional magnetic resonance data and the magnetic resonance diffusion tensor imaging data into the association perception dual-channel generation module to perform an interactive association perception fusion and obtain a multi-modal brain activity signal feature, a multi-modal effective connectivity matrix, and a reconstructed structural connectivity matrix; a prediction module, configure to input the multi-modal effective connectivity matrix into the disease feature regression module for prediction, input the reconstructed structural connectivity matrix into the topological structure discriminator for prediction, and input the multi-modal brain region activity signal feature into the time-space joint discriminator for prediction; and an updating module, configured to reversely update the association perception dual-channel generation module, the disease feature regression module, the topological structure discriminator, and the time-space joint discriminator, according to a predicted result and a pre-constructed loss function.

To resolve the foregoing technical problem, another technical solution used in this disclosure is that: providing a computer device, wherein the computer device comprises a processor and a memory coupled to the processor, wherein the memory stores program instructions, and when the program instructions are executed by the processor, the processor performs steps of any one of the above-mentioned the multi-modal brain network computation method associated with structural function.

To resolve the foregoing technical problem, another technical solution used in this disclosure is that: providing a storage medium, wherein the storage medium stores program instructions for implementing any one of the above-mentioned the multi-modal brain network computation method associated with structural function.

The beneficial effects of the present disclosure is that: the multi-modal brain network computation method associated with structural function of the present disclosure performs cross fusion on the brain functional magnetic resonance data and the magnetic resonance diffusion tensor imaging data by the association perception dual-channel generation module of the brain disease prediction model, to obtain the multi-modal brain activity signal feature, the multi-modal brain effective connectivity matrix, and the reconstructed structural connectivity matrix, which realizes the nonlinear multi-level fusion of multi-modal heterogeneous-heterogeneous data, and then uses the multi-modal brain region activity signal feature to perform the adversarial learning on the disease feature regression module, uses the multi-modal active connectivity matrix and the topological structure discriminator for adversarial learning, and uses the reconstructed structural connectivity matrix and the time-space joint discriminator for adversarial learning, to construct a multivariate collaborative generative adversarial strategy, comprehensively guides the learning of the model from the three aspects of time series signal of brain region activity, spatial field distribution and topological structure, realizes a bidirectional constraint on the functional state and intrinsic structure of the multi-modal effective connectivity, and greatly improves accuracy, robustness and generalization ability of the model.

### BRIEF DESCRIPTION OF DRAWINGS

FIG. 1 is a block diagram of a brain disease prediction model, according to an embodiment of the present disclosure.

FIG. 2 is a flowchart of a multi-modal brain network computation method associated with structural function, according to an embodiment of the present disclosure.

FIG. 3 is a block diagram of an association perception dual-channel generation module, according to an embodiment of the present disclosure.

FIG. 4 is a block diagram of a multi-modal brain network computation apparatus associated with structural function, according to an embodiment of the present disclosure.

FIG. 5 is a block diagram of a computer device, according to an embodiment of the present disclosure.

FIG. 6 is a block diagram of a storage medium, according to an embodiment of the present disclosure.

### DESCRIPTION OF EMBODIMENTS

The technical solutions in the embodiments of the present disclosure will be clearly and completely described hereinafter with reference to the accompanying drawings in the embodiments of the present application. Obviously, the described embodiments are only a part rather than all of the embodiments of the present disclosure. Based on the embodiments of the disclosure, all other embodiments obtained by a person of ordinary skill in the art without creative efforts fall within the protection scope of the present disclosure.

The terms “first”, “second”, and “third” in this application are used only for description purposes, and cannot be understood as indicating or implying relative importance or implying a quantity of indicated technical features. Therefore, a feature defined as “first”, “second”, and “third” may explicitly or implicitly include at least one feature. In the description of this application, “multiple” means at least two, for example, two or three, unless otherwise specifically limited. All directional indications (such as up, down, left, right, front, and back) in the embodiments of the present disclosure are only used to explain a relative positional relationship, a motion condition, and the like between components in a specific posture (as shown in the accompanying drawings). If the specific posture changes, the directional indication changes accordingly. In addition, the terms “include” and “have” and any variations thereof are intended to cover the inclusion of non-exclusive. For example, a process, method, system, product, or computer device that includes a series of steps or units is not limited to a listed step or unit, but optionally further includes an unlisted step or unit, or optionally further includes another step or unit inherent to the process, method, product, or computer device.

Referring to “embodiments” herein means that the specific features, structures, or features described with reference to the embodiments may be included in at least one embodiment of the present disclosure. That the phrase appears at various locations in the specification does not necessarily refer to a same embodiment, nor is it a separate or alternative embodiment mutually exclusive with another embodiment. A person skilled in the art explicitly and implicitly understands that the embodiments described in this specification may be combined with other embodiments.

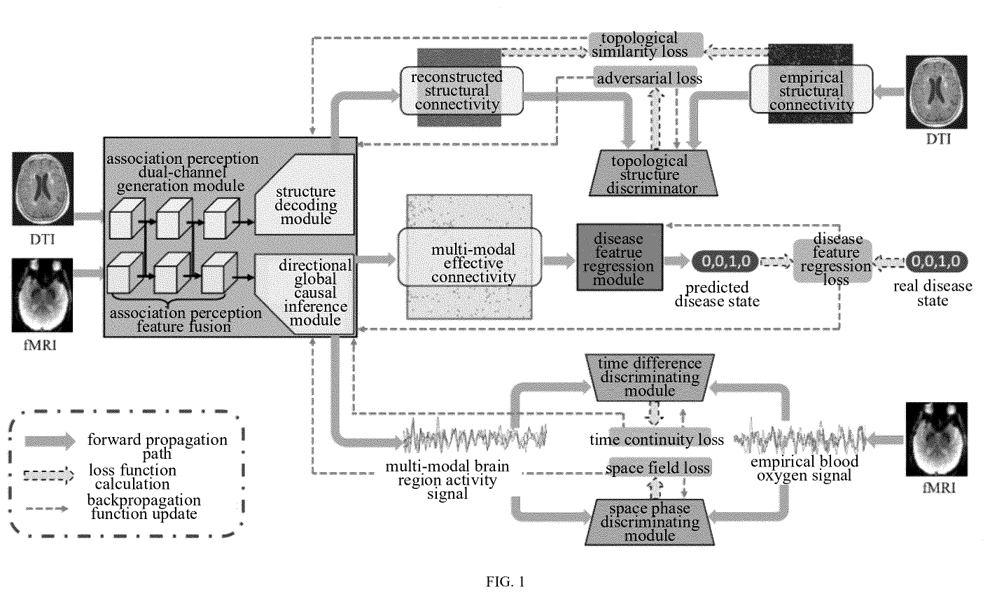

FIG. 1 is a block diagram of a brain disease prediction model, according to an embodiment of the present disclosure. As shown in FIG. 1, the brain disease prediction model includes an association perception dual-channel generation module, a disease feature regression module, a topological structure discriminator, and a time-space joint discriminator. Wherein, the association perception dual-channel generation module is configured to perform a multi-level non-linear mutual conversion on brain region image features extracted from brain functional magnetic resonance data and magnetic resonance diffusion tensor imaging data, and fuse brain region function information and physical neuron connection information of brain internal tissue. The disease feature regression module is configured to predict a probability that a patient may suffer from a tested brain disease, according to a multi-modal effective connectivity matrix output by the association perception two-channel generation module. The topological structure discriminator and the time-space joint discriminator are configured to comprehensively guide the model to learn from three aspects of time continuity, spatial field distribution, and topological structure of brain region activity time series signals, thereby realizing a bidirectional constraint on a functional state and an internal structure of the multi-modal brain effective connectivity.

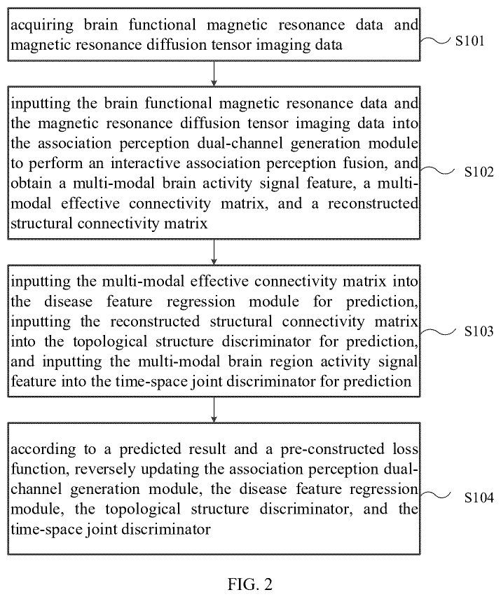

FIG. 2 is a flowchart of a multi-modal brain network computation method associated with structural function, according to an embodiment of the present disclosure. It should be noted that, if there is a substantially same result, the method in the present disclosure is not limited to the procedure sequence shown in FIG. 2. As shown in FIG. 2, the multi-modal brain network computation method associated with structural function includes the following steps:

S101, acquiring brain functional magnetic resonance data and magnetic resonance diffusion tensor imaging data.

Specifically, in this embodiment, pre-acquired brain functional magnetic resonance imaging (fMRI) data and magnetic resonance diffusion tensor imaging (DTI) data that are used as sample data to train the brain disease prediction model.

S102, inputting the brain functional magnetic resonance data and the magnetic resonance diffusion tensor imaging data into the association perception dual-channel generation module to perform an interactive association perception fusion and obtain a multi-modal brain activity signal feature, a multi-modal effective connectivity matrix, and a reconstructed structural connectivity matrix.

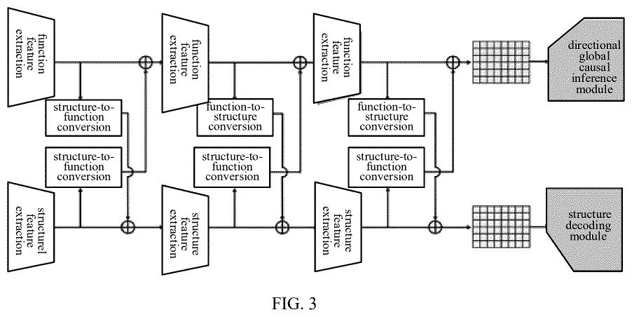

Specifically, referring to FIG. 3, the association perception dual-channel generation module includes a brain region feature extraction module, a structure-to-function conversion module, a function-to-structure conversion module, a directional global causal inference module, and a structure decoding module. The association perception dual-channel generation module can extract complementary information of different scales and layers in heterogeneous data by an alternating multi-layer interleaved structure, and perform a deep complementary information fusion in a manner of non-linear repeated interaction, thereby achieving an effect of efficient fusion of heterogeneous features.

Further, step S102 specifically includes:

1. Separately extracting a first initial feature and a second initial feature from the brain functional magnetic resonance data and the magnetic resonance diffusion tensor imaging data by the brain region feature extraction module.

Specifically, using the brain region feature extraction module to separately extract from the brain functional magnetic resonance data and the magnetic resonance diffusion tensor imaging data. Denoting the first initial feature extracted from the brain functional magnetic resonance data as X=(x₁, x₂, . . . , xₙ)∈ [symbol] ^{n×p}. Denoting the second initial feature extracted from the magnetic resonance diffusion tensor imaging data as Y=(y₁, y₂, . . . , yₙ)∈ [symbol] ^{n×q}. Wherein, n represents a number of brain regions, p and q represent vector dimensions of each brain region with respect to the first initial feature and the second initial feature, respectively.

2. Inputting the first initial feature and the second initial feature into the structure-to-function conversion module, performing a weighted fusion on a feature output from the structure-to-function conversion module and the first initial feature, to obtain a new first initial feature, and repeat this step to finally obtain the multi-modal brain region activity signal feature.

3. Inputting the first initial feature and the second initial feature into the function-to-structure conversion module, performing a weighted fusion on a feature output from the function-to-structure conversion module and the second initial feature to obtain a new second initial feature, and repeat this step to finally obtain the multi-modal structure feature.

In this embodiment, the first initial feature and the second initial feature are interconverted by the function-to-structure conversion module and the structure-to-function conversion module, and the above-mentioned two conversion modules are implemented based on association perception Transformer. Wherein, an output formula of the function-to-structure conversion module is as follows:

$$F 2 S ( X , Y ) = Attention ( q ( X ) , k ( y ) , v ( X ‖ η \cdot Y ) ) ;$$

Wherein, η represents a super parameter, a default value is 0.1, and a symbol ∥ represents feature association aggregation. q(·), k(·), v(·) are transformation functions formed by a neural network, that separately map X to feature space of dimension [symbol] ^{n×d}^{ₖ}, map Y to feature space of dimension [symbol] ^{n×d}^{ₖ}, and map X∥η·Y to feature space of dimension [symbol] ^{n×d}^{ᵥ}.

Similarly, an output formula of the structure-to-function conversion module is as follows:

$$S 2 F ( X , Y ) = Attention ( q ( Y ) , k ( X ) , v ( Y ‖ η \cdot X ) ) ;$$

Wherein, mapping X to the feature space of the dimension [symbol] ^{n×d}^{ₖ}, mapping Y to the feature space of the dimension [symbol] ^{n×d}^{ₖ}, and mapping Y∥η·X to the feature space of the dimension [symbol] ^{n×d}^{ᵥ}.

In this embodiment, a calculating formula of an attention mechanism is as follows:

$$Attention ( Q , K , V ) = softmax ( \frac{{QK}^{T}}{\sqrt{{d}_{k}}} ) V ;$$

Wherein, Q∈ [symbol] ^{n×d}^{ₖ}, K∈ [symbol] ^{n×d}^{ₖ}, V∈ [symbol] ^{n×d}^{ᵥ}.

Specifically, when the first initial feature and the second initial feature are input into the structure-to-function conversion module, and the feature output from the structure-to-the function conversion module is performed the weighted fusion with the first initial feature to obtain the new first initial feature. A weighted coefficient of the new first initial feature is set to 0.1. Similarly, the function-to-structure conversion module obtains the new second initial feature, thereby completing a first interactive association perception fusion. Then, the above-mentioned process is performed again on the new first initial feature and the new second initial feature, and after several times of the association perception fusion, the multi-modal brain region activity signal feature T=(t₁, t₂, . . . , tₙ) and the multi-modal brain structure feature D=(d₁, d₂, . . . , dₙ) are finally obtained.

4. Inputting the multi-modal brain region activity signal feature into the directional global causal inference module to obtain the multi-modal brain effective connectivity matrix, and inputting the multi-modal brain structure feature to the structure decoding module to obtain the reconstructed structural connectivity matrix.

The association perception dual-channel generation module in this embodiment implements the overall non-linear fusion of different-level features by the alternating multi-layer interleaved structure. Compared with a conventional cooperative fusion or a tower-type fusion, the alternating multi-layer interleaved structure provided in this embodiment can extract complementary information of different scales and layers in heterogeneous data, and perform the deep complementary information fusion in the manner of non-linear repeated interaction, thereby achieving an effect of efficient fusion of heterogeneous features.

S103, inputting the multi-modal effective connectivity matrix into the disease feature regression module for prediction, inputting the reconstructed structural connectivity matrix into the topological structure discriminator for prediction, and inputting the multi-modal brain region activity signal feature into the time-space joint discriminator for prediction.

Specifically, after obtaining the multi-modal brain effective connectivity matrix, the reconstructed structural connectivity matrix, and the multi-modal brain region activity signal feature, the multi-modal brain effective connectivity matrix, the multi-modal brain reconstructed structural connectivity matrix, and the multi-modal brain region activity signal feature are separately input into the disease feature regression module, the topological structure discriminator, and the time-space joint discriminator for adversarial learning. Wherein, the disease feature regression module is configured to predict a prediction probability of a tested brain disease. The topological structure discriminator and the time-space joint discriminator are configured to guide the model to learn from three aspects of a time continuity, a spatial field distribution, and a topological structure of the brain region activity time sequence signal.

Further, step S103 specifically includes:

1. Inputting the multi-modal brain effective connectivity matrix into the disease feature regression module for prediction, to obtain a disease state prediction probability.

Specifically, the disease feature regression module uses the multi-modal brain effective connectivity matrix as an input to output the prediction probability of the tested disease state. The disease feature regression module includes a feature sensor, an information aggregation layer, a higher-order feature extraction layer, an overall feature analysis layer, and a state probability prediction network, to finally obtain the probability of the tested disease state. By comparing with a known real disease state label, the disease feature regression module guides the association perception dual-channel generation module to learn.

2. Inputting the reconstructed structural connectivity matrix and an empirical structural connectivity matrix output by a pre-processing software template into the topological structure discriminator for prediction, to obtain a probability that the reconstructed structural connectivity matrix is output by the association perception dual-channel generation module or output by the pre-processing software template.

Specifically, the topological structure discriminator uses the empirical structural connectivity matrix output by the reconstructed structural connectivity matrix and the pre-processing software template as an input, and outputs a probability that the reconstructed structural connectivity matrix is output by the association perception dual-channel generation module or output by the pre-processing software template. Wherein, the topological structure discriminator includes a multi-layer nonlinear topological perception network and a full connection layer, and an update formula of the multi-layer nonlinear topological perception network is represented as follows:

$${F}^{( l + 1 )} = σ ( {D}^{- \frac{1}{2}} \cdot S \cdot {D}^{- \frac{1}{2}} {F}^{( l )} {W}^{( l )} + {b}^{( l )} ) ;$$

Wherein, S represents the reconstructed structural connectivity matrix, D represents a weighted dispersion matrix corresponding to the reconstructed structural connectivity matrix, F^{(l)} represents a topological feature of the lth layer, F^{(l+1)} represents a topological feature of the (l+1)th layer, W^{(l)} is a learnable weight matrix of the lth layer, b^{(l)} is a learnable nonlinear deviation of the lth layer, and σ represents a sigmoid activation function. Sigmoid is a library function of deep learning framework pytorch.

Specifically, the multi-layer nonlinear topological perception network perceives a homology relationship in a structure network by a graph topology iteration technology, directly and quantitatively calculates topological features of each order from the structural connectivity matrix. Compared with a feature extraction manner by using a multi-layer perceptron in a traditional method, the multi-layer nonlinear topological perception network proposed in this embodiment focuses on the learning of topological features, reduces interference of other irrelevant features, and can comprehensively and systematically characterize the structural connections from topological features more as a whole.

3. Inputting the multi-modal brain region activity signal feature and an empirical blood oxygen signal output by the pre-processing software template into the time-space joint discriminator for prediction, to obtain a probability that the multi-modal brain activity signal feature is output by the association perception dual-channel generation module or output by the pre-processing software template.

Wherein, the time-space joint discriminator includes a time difference discriminating module and a space phase discriminating module. The time difference discriminating module includes a time sequence second-order difference layer, an oscillation fitting layer, a nonlinear fusion layer, and a continuity analysis network. The space phase discriminating module includes a phase perception layer, a field strength detection layer, a field action path calculation layer, a nonlinear fusion layer, and a field distribution prediction layer. The time difference discriminating module is configured to constrain the association perception dual-channel generation module from a time continuity feature of the brain region activity time series signal, and the space phase discriminating module is configured to constrain the association perception dual-channel generation module from a field distribution of the brain region activity signal, thereby realizing a bidirectional constraint on the functional state and the intrinsic structure of the multi-modal brain effective connectivity matrix.

Specifically, the time difference discriminating module and the spatial phase discriminating module use the multi-modal brain region activity signal feature and the empirical blood oxygen signal output by the pre-processing software template as an input, and output the probability that the multi-modal brain region activity signal feature is output by the association perception dual-channel generation module or output by the pre-processing software template.

S104, according to a predicted result and a pre-constructed loss function, reversely updating the association perception dual-channel generation module, the disease feature regression module, the topological structure discriminator, and the time-space joint discriminator.

Wherein, the loss function includes a disease feature regression loss, a topological adversarial loss, a topological perception loss, a time-space joint adversarial loss, and an attribution metric constraint loss.

The disease feature regression loss is constructed based on the Kullback-Leibler divergence, and is configured to guide the disease feature regression module and the association perception dual-channel generation module to update parameters, and which is represented as:

$${ℒ}_{cls} = {𝔼}_{y} [ - log {p}_{c} ( y \vert A ) ] ;$$

Wherein, [symbol] _{cls} represents the disease feature regression loss, A represents the multi-modal effective connectivity matrix, y represents disease states, taking Alzheimer's disease as an example, the disease states includes healthy control group, mild cognitive impairment, advanced cognitive impairment, and Alzheimer's disease, p_{c}(y|A) represents the disease state prediction probability, [symbol] _{y} represents an expectation of the disease state probability predicted by the model in a real label distribution, and is used as a loss function for guiding the model to learn.

The topological adversarial loss is configured to guide the topological structure discriminator and the association perception dual-channel generation module to update parameters, and the topological adversarial loss is represented as:

$${ℒ}_{top}^{D} = 𝔼 [ log {D}_{top} ( {S}^{′} ) ] + 𝔼 [ log ( 1 - {D}_{top} ( S ) ) ] ;$$

$${ℒ}_{top}^{G} = 𝔼 [ log {D}_{top} ( S ) ] ;$$

Wherein, [symbol] ₜₒₚ^{D} represents a loss function that guides the topological structure discriminator to learn, [symbol] ₜₒₚ^{G} represents a loss function that guides the generator to learn by the topological structure discriminator, S represents the reconstructed structural connectivity matrix, S′ represents the empirical structural connectivity matrix that is output by the pre-processing software template, and Dₜₒₚ represents the topological structure discriminator.

To better acquire a higher-order topological difference between the reconstructed structural connectivity and the empirical structural connectivity, in this embodiment, the topological perception loss is designed to guide the association perception dual-channel module to update parameters, which is represented as:

$${ℒ}_{awa} = {‖ S - {S}^{′} ‖}_{2} + λ {‖ {SS}^{T} - ( {S}^{′} ) {( {S}^{′} )}^{T} ‖}_{2} ;$$

Wherein, [symbol] _{awa} represents the topological perception loss, ∥·∥₂ represents a Frobenius norm of a matrix, and λ represents a preset super parameter. In this embodiment, a structure decoding module and a topology perception loss are provided. Compared with a conventional adversarial learning method that directly compares brain region features, the topological perception loss characterizes a low-order-high-order comprehensive topological difference between a reconstructed structure brain network and an empirical structure brain network obtained by the pre-processing software template. After the model training is completed, no professional physician is required to label the model, and the model can accurately learn the topological structure feature in multi-modal brain data, thereby improving an accuracy of the multi-modal brain effective connectivity calculation.

The time-space joint adversarial loss is configured to guide the time difference discriminating module, the space phase discriminating module, and the association perception dual-channel generation module to update parameters, and which is represented as:

$${ℒ}_{uni}^{D} = 𝔼 [ log ( \frac{1}{2} {D}_{tmp} ( {S}^{′} ) + \frac{1}{2} {D}_{spa} ( {S}^{′} ) ) ] + 𝔼 [ log ( 1 - \frac{1}{2} {D}_{tmp} ( S ) - \frac{1}{2} {D}_{spa} ( S ) ] ;$$

$${ℒ}_{uni}^{G} = 𝔼 [ log ( \frac{1}{2} {D}_{tmp} ( S ) + \frac{1}{2} {D}_{spa} ( S ) ) ] ;$$

Wherein, [symbol] ᵤₙᵢ^{D} represents a loss function for guiding the time-space joint discriminator to learn, [symbol] ᵤₙᵢ^{G} represents a loss function for guiding the generator to learn by the time-space joint discriminator, and aims to enable the multi-modal brain region activity signal generated by the model to learn the time-frequency distribution of blood oxygen level dependence extracted from fMRI data. Dₜₘₚ represents the time difference discriminating module, and Dₛₚₐ represents the space phase discriminating module. According to the time-space joint adversarial loss provided in this embodiment, the time continuity feature and the time spatial field distribution of the multi-modal brain region activity series are integrated into an organic whole. By introducing the second-order difference layer and the oscillation fitting layer to characterize the continuity feature of the time series, and by using the phase perception layer, the field strength detection layer, and the field action path calculation layer to characterize the spatial field distribution of the brain region activity, the two jointly constrain the adversarial learning and solve a problem of a low accuracy of a traditional generative adversarial strategy in time series generation and extraction.

It should be understood that the effective connectivity characterizes a causal relationship between the activity signals of various brain regions and satisfies the directional overall constraint of the structural equation tᵢ=Σⱼ₌₁ⁿ A_{i,j}tⱼ+∈ᵢ, wherein ∈ᵢ represents noise. Based on the structural equation, this embodiment designs the attribution metric constraint loss to constrain the multi-modal effective connectivity matrix and the multimodal activity signal features learned by the model. The attribution metric constraint loss is configured to guide the association perception dual-channel generation module to update parameters, and which is represented as:

$$ℒ ( A , B ) = \vert \vert A \cdot AB - 2 AB + B {\vert \vert}_{2} ;$$

Wherein, [symbol] (A,B) represents the attributive metric constraint loss, and B represents the multi-modal brain region activity signal feature. In the association perception dual-channel generation module in this embodiment, based on the brain region activity signal structure equation theory in the effective connectivity, the directional global causal inference module and the attribution metric constraint loss are designed. Compared with a conventional effective connectivity calculation model which directly calculates the effective connectivity, the association perception dual-channel generation module provided in this embodiment simultaneously outputs the multi-modal effective connectivity matrix and the multi-modal brain activity signal feature. Meanwhile, the association perception dual-channel generation module constraints an internal relationship between the two by the attribution metric constraint loss. Thus, an output precision of the brain region disease prediction model obtained in this embodiment is higher than the conventional prediction model, and has stronger interpretability.

The multi-modal brain network computation method associated with structural function in this embodiment, performs cross-fusion on the brain functional magnetic resonance data and the magnetic resonance diffusion tensor imaging data by the association perception dual-channel generation module of the brain disease prediction model, to obtain the multi-modal brain activity signal feature, the multi-modal brain effective connectivity matrix, and the reconstructed structural connectivity matrix, which realizes the nonlinear multi-level fusion of multi-modal heterogeneous-heterogeneous data, and then uses the multi-modal brain region activity signal feature to perform the adversarial learning on the disease feature regression module, uses the multi-modal active connectivity matrix and the topological structure discriminator for adversarial learning, and uses the reconstructed structural connectivity matrix and the time-space joint discriminator for adversarial learning, to construct a multivariate collaborative generative adversarial strategy, comprehensively guides the learning of the model from the three aspects of time series signal of brain region activity, spatial field distribution and topological structure, realizes a bidirectional constraint on the functional state and intrinsic structure of the multi-modal effective connectivity, and greatly improves accuracy, robustness and generalization ability of the model.

Further, after the multi-modal brain network associated with the structural function is calculated, the brain disease prediction may be performed by the brain disease prediction model. A method for predicting the brain disease by the brain disease prediction model includes:

1. Acquiring brain functional magnetic resonance data and magnetic resonance diffusion tensor imaging data of a patient.

2. Inputting the brain functional magnetic resonance data and the magnetic resonance diffusion tensor imaging data into the association perception dual-channel generation module to perform an interactive association perception fusion to obtain a multi-modal brain effective connectivity matrix.

3. Inputting the multi-modal brain effective connectivity matrix into the disease feature regression module to predict, thereby obtaining a prediction probability that the patient a brain disease.

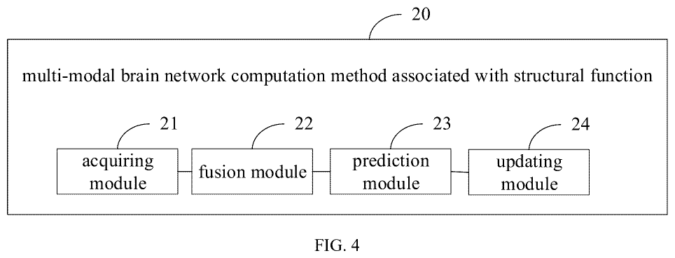

FIG. 4 is a block diagram of a multi-modal brain network computation apparatus associated with structural function, according to an embodiment of the present disclosure. Referring to FIG. 4, the multi-modal brain network computation apparatus associated with structural function 20 includes an acquiring module 21, a fusion module 22, a prediction module 23, and an updating module 24.

The acquiring module 21, is configured to acquire brain functional magnetic resonance data and magnetic resonance diffusion tensor imaging data.

The fusion module 22, is configured to input the brain functional magnetic resonance data and the magnetic resonance diffusion tensor imaging data into the association perception dual-channel generation module to perform an interactive association perception fusion and obtain a multi-modal brain activity signal feature, a multi-modal effective connectivity matrix, and a reconstructed structural connectivity matrix.

The prediction module 23, is configure to input the multi-modal effective connectivity matrix into the disease feature regression module for prediction, input the reconstructed structural connectivity matrix into the topological structure discriminator for prediction, and input the multi-modal brain region activity signal feature into the time-space joint discriminator for prediction.

The updating module 24, is configured to reversely update the association perception dual-channel generation module, the disease feature regression module, the topological structure discriminator, and the time-space joint discriminator, according to a predicted result and a pre-constructed loss function.

The updating module 24, is configured to reversely update the association perception dual-channel generation module, the disease feature regression module, the topological structure discriminator, and the time-space joint discriminator, according to a predicted result and a pre-constructed loss function.

Optionally, the association perception dual-channel generation module includes a brain region feature extraction module, a structure-to-function conversion module, a function-to-structure conversion module, a directional global causal inference module, and a structure decoding module.

Optionally, the fusion module 22 performs to input the brain functional magnetic resonance data and the magnetic resonance diffusion tensor imaging data into the association perception dual-channel generation module to perform an interactive association perception fusion and obtain a multi-modal brain activity signal feature, a multi-modal effective connectivity matrix, and a reconstructed structural connectivity matrix, includes: separately extract a first initial feature and a second initial feature from the brain functional magnetic resonance data and the magnetic resonance diffusion tensor imaging data by the brain region feature extraction module; and input the first initial feature and the second initial feature into the structure-to-function conversion module, perform a weighted fusion on a feature output from the structure-to-function conversion module and the first initial feature, to obtain a new first initial feature, and repeat this step to finally obtain the multi-modal brain region activity signal feature; and input the first initial feature and the second initial feature into the function-to-structure conversion module, perform a weighted fusion on a feature output from the function-to-structure conversion module and the second initial feature to obtain a new second initial feature, and repeat this step to finally obtain the multi-modal structure feature; and input the multi-modal brain region activity signal feature into the directional global causal inference module to obtain the multi-modal brain effective connectivity matrix, and input the multi-modal brain structure feature to the structure decoding module to obtain the reconstructed structural connectivity matrix.

Optionally, the prediction module 23 performs to input the multi-modal brain effective connectivity matrix into the disease feature regression module for prediction, input the reconstructed structural connectivity matrix into the topological structure discriminator for prediction, and input the multi-modal brain region activity signal feature into the time-space joint discriminator for prediction, includes: input the multi-modal brain effective connectivity matrix into the disease feature regression module for prediction, to obtain a disease state prediction probability; and input the reconstructed structural connectivity matrix and an empirical structural connectivity matrix output by a pre-processing software template into the topological structure discriminator for prediction, to obtain a probability that the reconstructed structural connectivity matrix is output by the association perception dual-channel generation module or output by the pre-processing software template; and input the multi-modal brain activity signal feature and an empirical blood oxygen signal output by the pre-processing software template into the time-space joint discriminator for prediction, to obtain a probability that the multi-modal brain activity signal feature is output by the association perception dual-channel generation module or output by the pre-processing software template.

Optionally, the time-space joint discriminator includes a time difference discriminating module and a spatial phase discriminating module, the time difference discriminating module is configured to constrain the association perception dual-channel generation module from a time continuity feature of a brain region activity time series signal, and the spatial phase discriminating module constrains the association perception dual-channel generation module from a spatial field distribution of the brain region activity signal.

Optionally, the loss function includes a disease feature regression loss, a topological adversarial loss, a topological perception loss, a time-space joint adversarial loss, and an attribution measure constraint loss;

The disease feature regression loss is configured to guide the disease feature regression module and the association perception dual-channel generation module to update parameters, and the disease feature regression loss is represented as:

$${ℒ}_{cls} = {𝔼}_{y} [ - log {p}_{c} ( y \vert A ) ] ;$$

Wherein, [symbol] _{cls} represents the disease feature regression loss, A represents the multi-modal effective connectivity matrix, y represents disease states, taking Alzheimer's disease as an example, the disease states includes healthy control group, mild cognitive impairment, advanced cognitive impairment, and Alzheimer's disease, p_{c}(y|A) represents the disease state prediction probability, [symbol] _{y} represents an expectation of the disease state probability predicted by the model in a real label distribution, and is used as a loss function for guiding the model to learn.

The topological adversarial loss is configured to guide the topological structure discriminator and the association perception dual-channel generation module to update parameters, and the topological adversarial loss is represented as:

$${ℒ}_{top}^{D} = 𝔼 [ log {D}_{top} ( {S}^{′} ) ] + 𝔼 [ log ( 1 - {D}_{top} ( S ) ) ] ;$$

$${ℒ}_{top}^{G} = 𝔼 [ log {D}_{top} ( S ) ] ;$$

Wherein, [symbol] ₜₒₚ^{D} represents a loss function that guides the topological structure discriminator to learn, [symbol] ₜₒₚ^{G} represents a loss function that guides the generator to learn by the topological structure discriminator, S represents the reconstructed structural connectivity matrix, S′ represents the empirical structural connectivity matrix that is output by the pre-processing software template, and Dₜₒₚ represents the topological structure discriminator.

The topological perception loss is configured to guide the association perception dual-channel generation module to update parameters, and the topological perception loss is represented as:

$${ℒ}_{awa} = {‖ S - {S}^{′} ‖}_{2} + λ {‖ {SS}^{T} - ( {S}^{′} ) {( {S}^{′} )}^{T} ‖}_{2} ;$$

Wherein, [symbol] _{awa} represents the topological perception loss, ∥·∥₂ represents a Frobenius norm of a matrix, and λ represents a preset super parameter.

The time-space joint adversarial loss is configured to guide the time difference discriminating module, the space phase discriminating module, and the association perception dual-channel generation module to update parameters, and the time-space joint adversarial loss is represented as:

$${ℒ}_{uni}^{D} = 𝔼 [ log ( \frac{1}{2} {D}_{tmp} ( {S}^{′} ) + \frac{1}{2} {D}_{spa} ( {S}^{′} ) ) ] + 𝔼 [ log ( 1 - \frac{1}{2} {D}_{tmp} ( S ) - \frac{1}{2} {D}_{spa} ( S ) ]$$

$${ℒ}_{uni}^{G} = 𝔼 [ log ( \frac{1}{2} {D}_{tmp} ( S ) + \frac{1}{2} {D}_{spa} ( S ) ) ] ;$$

wherein, [symbol] ᵤₙᵢ^{D} represents a loss function for guiding the time-space joint discriminator to learn, [symbol] ᵤₙᵢ^{G} represents a loss function for guiding the generator to learn by the time-space joint discriminator, and aims to enable the multi-modal brain region activity signal generated by the model to learn the time-frequency distribution of blood oxygen level dependence extracted from fMRI data. Dₜₘₚ represents the time difference discriminating module, and Dₛₚₐ represents the space phase discriminating module.

The attribution metric constraint loss is configured to guide the association perception dual-channel generation module to update parameters, and is represented as:

$$ℒ ( A , B ) = \vert \vert A \cdot AB - 2 AB + B {\vert \vert}_{2}$$

Wherein, [symbol] (A,B) represents the attributive metric constraint loss, and B represents the multi-modal brain region activity signal feature.

Optically, the topological structure discriminator includes a multi-layer nonlinear topological perception network and a fully connection layer, and an updating formula of the multi-layer nonlinear topological perception network is represented as:

$${F}^{( l + 1 )} = σ ( {D}^{- \frac{1}{2}} \cdot S \cdot {D}^{- \frac{1}{2}} {F}^{( l )} {W}^{( l )} + {b}^{( l )} ) ;$$

Wherein, S represents the reconstructed structural connectivity matrix, D represents a weighted dispersion matrix corresponding to the reconstructed structural connectivity matrix, F^{(l)} represents a topological feature of the lth layer, F^{(l+1)} represents a topological feature of the (l+1)th layer, W^{(l)} is a learnable weight matrix of the lth layer, b^{(l)} is a learnable nonlinear deviation of the lth layer, and σ represents a sigmoid activation function. Sigmoid is a library function of deep learning framework pytorch.

For other details of the technical solutions of the modules in the above-mentioned multi-mode brain network computation apparatus, refer to descriptions in the multi-mode brain network computation method in the above-mentioned embodiment. Details are omitted herein.

It should be noted that each embodiment in this specification is described in a progressive manner. Each embodiment focuses on a difference from another embodiment. For a same similar part of the embodiments, refer to each other. For an apparatus embodiment, because the apparatus embodiment is basically similar to the method embodiment, description is relatively simple. For related parts, refer to partial descriptions of the method embodiment.

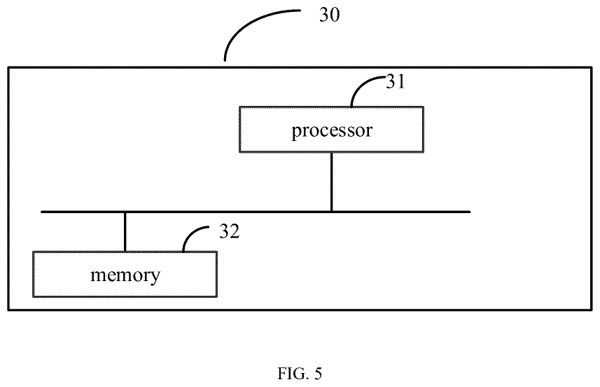

Referring to FIG. 5, FIG. 5 is a block diagram of a computer device according to an embodiment of the present disclosure. As shown in FIG. 5, the computer device 30 includes a processor 31 and a memory 32 coupled to the processor 31. The memory 32 stores program instructions. When the program instructions are executed by the processor 31, the processor 31 performs the steps of the multi-modal brain network computation method described in any one of the foregoing embodiments.

The processor 31 may also be referred to as a CPU (Central Processing Unit, Central Processing Unit). The processor 52 may be an integrated circuit chip, and have a signal processing capability. The processor 52 may further be a general-purpose processor, a digital signal processor (DSP), an application-specific integrated circuit (ASIC), a field programmable gate array (FPGA) or another programmable logic device, a discrete gate or transistor logic device, or a discrete hardware component. The general-purpose processor may be a microprocessor, or the processor may be any conventional processor or the like.

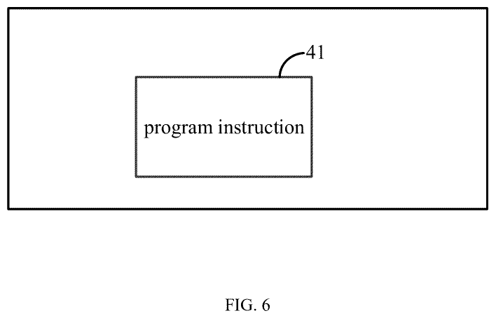

Referring to FIG. 6, FIG. 6 is a block diagram of a storage medium, according to an embodiment of the present disclosure. The storage medium in this embodiment of the present invention stores program instructions 41 that can implement the foregoing multi-modal brain network calculation method. The program instructions 41 may be stored in the foregoing storage medium in a form of a software product, and includes several instructions for instructing a computer device (which may be a personal computer, a server, or a network device) or a processor (processor) to perform all or a part of the steps of the methods in the implementation manners of this disclosure. The foregoing storage medium includes any medium that can store program code, such as a USB flash drive, a removable hard disk, a read-only memory (ROM, Read-Only Memory), a random-access memory (RAM, Random Access Memory), a magnetic disk, or an optical disc, or a computer device such as a computer, a server, a mobile phone, or a tablet.

In the embodiments provided in the present disclosure, it should be understood that the disclosed system, apparatus, and method may be implemented in another manner. For example, the described system embodiment is merely an example. For example, unit division is merely logical function division. In actual implementation, there may be another division manner. For example, multiple units or components may be combined or integrated into another system, or some features may be ignored or not performed. On the other hand, the displayed or discussed mutual coupling or direct coupling or communication connection may be through some interfaces, indirect coupling or communication connection of the apparatus or unit, and may be in an electrical, mechanical, or other form.

In addition, functional units in the embodiments of the present disclosure may be integrated into one processing unit, or each unit may exist separately physically, or two or more units may be integrated into one unit. The foregoing integrated unit may be implemented in a form of hardware, or may be implemented in a form of a software functional unit. The foregoing descriptions are merely implementations of the present disclosure, and are not intended to limit the scope of the present disclosure. Any equivalent structure or equivalent procedure transformation performed by using the content in the specification and accompanying drawings of the present disclosure, or directly or indirectly applied in another related technical field is included in the protection scope of the present disclosure.

What is claimed is:

1. A multi-modal brain network computation method associated with structural function, wherein the method is applied to train a brain disease prediction model, and the brain disease prediction model comprises an association perception dual-channel generation module, a disease feature regression module, a topological structure discriminator, and a time-space joint discriminator; the method comprising:
acquiring brain functional magnetic resonance data and magnetic resonance diffusion tensor imaging data;
inputting the brain functional magnetic resonance data and the magnetic resonance diffusion tensor imaging data into the association perception dual-channel generation module to perform an interactive association perception fusion, and obtain a multi-modal brain activity signal feature, a multi-modal effective connectivity matrix, and a reconstructed structural connectivity matrix;
inputting the multi-modal effective connectivity matrix into the disease feature regression module for prediction, inputting the reconstructed structural connectivity matrix into the topological structure discriminator for prediction, and inputting the multi-modal brain region activity signal feature into the time-space joint discriminator for prediction; and
according to a predicted result and a pre-constructed loss function, reversely updating the association perception dual-channel generation module, the disease feature regression module, the topological structure discriminator, and the time-space joint discriminator.

2. The multi-modal brain network computation method associated with structural function according to claim 1, wherein the association perception dual-channel generation module comprises a brain region feature extraction module, a structure-to-function conversion module, a function-to-structure conversion module, a directional global causal inference module, and a structure decoding module.

3. The multi-modal brain network computation method associated with structural function according to claim 2, wherein inputting the brain functional magnetic resonance data and the magnetic resonance diffusion tensor imaging data into the association perception dual-channel generation module to perform an interactive association perception fusion and obtain a multi-modal brain activity signal feature, a multi-modal effective connectivity matrix, and a reconstructed structural connectivity matrix, comprises:
separately extracting a first initial feature and a second initial feature from the brain functional magnetic resonance data and the magnetic resonance diffusion tensor imaging data by the brain region feature extraction module;
inputting the first initial feature and the second initial feature into the structure-to-function conversion module, performing a weighted fusion on a feature output from the structure-to-function conversion module and the first initial feature, to obtain a new first initial feature, and repeat this step to finally obtain the multi-modal brain region activity signal feature;
inputting the first initial feature and the second initial feature into the function-to-structure conversion module, performing a weighted fusion on a feature output from the function-to-structure conversion module and the second initial feature, to obtain a new second initial feature, and repeat this step to finally obtain the multi-modal structure feature; and
inputting the multi-modal brain region activity signal feature into the directional global causal inference module to obtain the multi-modal brain effective connectivity matrix, and inputting the multi-modal brain structure feature to the structure decoding module to obtain the reconstructed structural connectivity matrix.

4. The multi-modal brain network computation method associated with structural function according to claim 1, wherein inputting the multi-modal brain effective connectivity matrix into the disease feature regression module for prediction, inputting the reconstructed structural connectivity matrix into the topological structure discriminator for prediction, and inputting the multi-modal brain region activity signal feature into the time-space joint discriminator for prediction, comprises:
inputting the multi-modal brain effective connectivity matrix into the disease feature regression module for prediction, to obtain a disease state prediction probability;
inputting the reconstructed structural connectivity matrix and an empirical structural connectivity matrix output by a pre-processing software template into the topological structure discriminator for prediction, to obtain a probability that the reconstructed structural connectivity matrix is output by the association perception dual-channel generation module or output by the pre-processing software template; and
inputting the multi-modal brain activity signal feature and an empirical blood oxygen signal output by the pre-processing software template into the time-space joint discriminator for prediction, to obtain a probability that the multi-modal brain activity signal feature is output by the association perception dual-channel generation module or output by the pre-processing software template.

5. The multi-modal brain network computation method associated with structural function according to claim 4, wherein the time-space joint discriminator comprises a time difference discriminating module and a spatial phase discriminating module, the time difference discriminating module is configured to constraint the association perception dual-channel generation module from a time continuity feature of a brain region activity time series signal, and the spatial phase discriminating module is configured to constraint the association perception dual-channel generation module from a spatial field distribution of the brain region activity signal.

6. The multi-modal brain network computation method associated with structural function according to claim 5, wherein the loss function comprises a disease feature regression loss, a topological adversarial loss, a topological perception loss, a time-space joint adversarial loss, and an attribution measure constraint loss;
the disease feature regression loss is configured to guide the disease feature regression module and the association perception dual-channel generation module to update parameters, and the disease feature regression loss is represented as:
$${ℒ}_{cls} = {𝔼}_{y} [ - log {p}_{c} ( y \vert A ) ] ;$$
wherein, [symbol] _{cls} represents the disease feature regression loss, A represents the multi-modal effective connectivity matrix, y represents a disease state, p_{c}(y|A) represents the disease state prediction probability, [symbol] y represents an expectation of the disease state probability predicted by the model in a real label distribution, and is used as a loss function for guiding the model to learn;
the topological adversarial loss is configured to guide the topological structure discriminator and the association perception dual-channel generation module to update parameters, and the topological adversarial loss is represented as:
$${ℒ}_{top}^{D} = 𝔼 [ log {D}_{top} ( {S}^{′} ) ] + 𝔼 [ log ( 1 - {D}_{top} ( S ) ) ] ;$$
$${ℒ}_{top}^{G} = 𝔼 [ log {D}_{top} ( S ) ] ;$$
wherein, [symbol] ₜₒₚ^{D} represents a loss function that guides the topological structure discriminator to learn, [symbol] ₜₒₚ^{G} represents a loss function that guides the generator to learn by the topological structure discriminator, S represents the reconstructed structural connectivity matrix, S′ represents the empirical structural connectivity matrix that is output by the pre-processing software template, and Dₜₒₚ represents the topological structure discriminator;
the topological perception loss is configured to guide the association perception dual-channel generation module to update parameters, and the topological perception loss is represented as:
$${ℒ}_{awa} = {‖ S - {S}^{′} ‖}_{2} + λ {‖ {SS}^{T} - ( {S}^{′} ) {( {S}^{′} )}^{T} ‖}_{2} ;$$
wherein, [symbol] _{awa} represents the topological perception loss, ∥·∥₂ represents a Frobenius norm of a matrix, and λ represents a preset super parameter;
the time-space joint adversarial loss is configured to guide the time difference discriminating module, the space phase discriminating module, and the association perception dual-channel generation module to update parameters, and the time-space joint adversarial loss is represented as:
$${ℒ}_{uni}^{D} = 𝔼 [ log ( \frac{1}{2} {D}_{tmp} ( {S}^{′} ) + \frac{1}{2} {D}_{spa} ( {S}^{′} ) ) ] + 𝔼 [ log ( 1 - \frac{1}{2} {D}_{tmp} ( S ) - \frac{1}{2} {D}_{spa} ( S ) ] ;$$
$${ℒ}_{uni}^{G} = 𝔼 [ log ( \frac{1}{2} {D}_{tmp} ( S ) + \frac{1}{2} {D}_{spa} ( S ) ) ] ;$$
wherein, [symbol] ᵤₙᵢ^{D} represents a loss function for guiding the time-space joint discriminator to learn, [symbol] ᵤₙᵢ^{G} represents a loss function for guiding the generator to learn by the time-space joint discriminator, Dₜₘₚ represents the time difference discriminating module, and Dₛₚₐ represents the space phase discriminating module;
the attributive metric constraint loss is configured to guide the association perception two-channel generation module to update parameters, and the attributive metric constraint loss is represented as:
$$ℒ ( A , B ) = \vert \vert A \cdot AB - 2 AB + B {\vert \vert}_{2} ;$$
wherein, [symbol] (A,B) represents the attributive metric constraint loss, B represents the multi-modal brain region activity signal feature.

7. The multi-modal brain network computation method associated with structural function according to claim 1, wherein the topological structure discriminator comprises a multi-layer nonlinear topological perception network and a fully connection layer, and an updating formula of the multi-layer nonlinear topological perception network is represented as:
$${F}^{( l + 1 )} = σ ( {D}^{- \frac{1}{2}} \cdot S \cdot {D}^{- \frac{1}{2}} {F}^{( l )} {W}^{( l )} + {b}^{( l )} ) ;$$
wherein, S represents the reconstructed structural connectivity matrix, D represents a weighted dispersion matrix corresponding to the reconstructed structural connectivity matrix, F^{(l)} represents a topological feature of the lth layer, F^{(l+1)} represents a topological feature of the (l+1)th layer, W^{(l)} is a learnable weight matrix of the lth layer, b^{(l)} is a learnable nonlinear deviation of the lth layer, and σ represents a sigmoid activation function.

8. A multi-modal brain network computation apparatus associated with structural function, comprising:
an acquiring module, configured to acquire brain functional magnetic resonance data and magnetic resonance diffusion tensor imaging data;
a fusion module, configured to input the brain functional magnetic resonance data and the magnetic resonance diffusion tensor imaging data into the association perception dual-channel generation module to perform an interactive association perception fusion and obtain a multi-modal brain activity signal feature, a multi-modal effective connectivity matrix, and a reconstructed structural connectivity matrix;
a prediction module, configure to input the multi-modal effective connectivity matrix into the disease feature regression module for prediction, input the reconstructed structural connectivity matrix into the topological structure discriminator for prediction, and input the multi-modal brain region activity signal feature into the time-space joint discriminator for prediction; and
an updating module, configured to reversely update the association perception dual-channel generation module, the disease feature regression module, the topological structure discriminator, and the time-space joint discriminator, according to a predicted result and a pre-constructed loss function.

9. A computer device, comprising a processor and a memory coupled to the processor, wherein the memory stores program instructions, and when the program instructions are executed by the processor, the processor performs steps of the multi-modal brain network computation method associated with structural function according to claim 1.

10. A non-transitory storage medium, wherein the non-transitory storage medium stores program instructions for implementing the multi-modal brain network computation method associated with structural function according to claim 1.
